(19) United States
(12) Patent Application Publication
(10) Pub. No.: US 2024/0260549 A1
(43) Pub. Date: Aug. 8, 2024

(54) METHODS AND SYSTEMS FOR AQUACULTURE

(71) Applicant: Vertical Oceans Ptd. Ltd., Singapore (SG)

(72) Inventors: Enzo ACERBI, Singapore (SG); John DIENER, Singapore (SG)

(21) Appl. No.: 18/411,545

(22) Filed: Jan. 12, 2024

Related U.S. Application Data
(63) Continuation of application No. PCT/SG22/50492, filed on Jul. 14, 2022.
(60) Provisional application No. 63/222,070, filed on Jul. 15, 2021.

(30) Foreign Application Priority Data
Jul. 14, 2022 (WO) PCT/SG22/50492

(51) Int. Cl. A01K 61/10; A01G 33/00; A01K 61/59

(52) U.S. Cl. CPC A01K 61/10; A01G 33/00; A01K 61/59

(57) ABSTRACT

Methods and systems for growing water-growing organisms are disclosed herein. The methods and systems may comprise use of a self-contained unit. A self-contained unit may be configured for the growth of at least one water-growing organism.

### CROSS-REFERENCE

This application is a continuation of International Patent Application No. PCT/SG22/50492, filed Jul. 14, 2022, which claims the benefit of U.S. Provisional Application No. 63/222,070, filed Jul. 15, 2021, each of which is incorporated herein by reference in its entirety.

### BACKGROUND

Commercial fishing and wild-caught seafood accounts for over 80% of seafood consumed in the United States. These methods can cause overfishing and population collapse of wild fish stocks. Additionally, environmental impacts caused by transporting seafood from its point of capture to inland markets can compound the negative effects that are seen from wild-caught seafood.

### SUMMARY

Recognized herein is a need for farmed seafood with a low environmental and spatial footprint. By combining farmed seafoods with vertical stacking, seafood can be grown local to its consumption, improving both the quality of the seafood as well as the price and environmental impact.

In an aspect, the present disclosure provides a self-contained unit, comprising: (a) a region configured for growth of at least one water-growing organism, wherein the growth is at a feed conversion rate of at most about 1. In another aspect, the present disclosure provides a self-contained unit, comprising: (a) a region configured for growth of at least one water-growing organism, wherein the growth is at a feed conversion rate of at most about 1.3.

In some embodiments, the self-contained unit further comprises an umbilical portion configured for delivery of one or more of electrical energy, gasses, solids, liquids, or any combination thereof. In some embodiments, the self-contained unit comprises a plurality of water-growing organisms. In some embodiments, a water-growing organism of the water-growing organisms is a water-growing plant. In some embodiments, the water-growing plant comprises an algae or a seaweed. In some embodiments, a water-growing organism of the water-growing organisms is a water-growing animal. In some embodiments, the water-growing animal comprises a fish or a shrimp. In some embodiments, a water-growing organism of the water-growing organisms is configured to subsist on waste generated by a second water-growing organism of the water-growing organisms. In some embodiments, the self-contained unit further comprises a lid configured to contain the region, wherein the lid is an insulated lid. In some embodiments, the insulated lid further comprises a heater or cooler. In some embodiments, the heater or the cooler is configured to maintain the temperature of water within the region at a temperature of at most about 1° C. from a set point temperature. In some embodiments, the heater or the cooler is configured to maintain the temperature of water within the region at a temperature of at most about 2° C. from a set point temperature. In some embodiments, the self-contained unit further comprises a nitrification moving bed bioreactor in fluid communication with the self-contained unit, which nitrification moving bed bioreactor comprises a containment vessel configured to enclose biofiltration media and a fluidizing aeration source. In some embodiments, the biofiltration media comprises a surface area of at least about 1000 square meters per gram (m²/g). In some embodiments, the biofiltration media comprises a surface area of at least about 900 square meters per cubic meter (m²/m³). In some embodiments, the biofiltration media comprises an oxygen concentration of at least about 9 parts per million (ppm). In some embodiments, the biofiltration media comprises an oxygen concentration of at least about 4 milligrams per liter (mg/L). In some embodiments, the biofiltration media has a neutral buoyancy. In some embodiments, the containment vessel has a volume of at most about 3 cubic meters (m³). In some embodiments, the containment vessel is substantially opaque. In some embodiments, the containment vessel is configured to permit flow of water through the containment vessel. In some embodiments, the nitrification moving bed bioreactor is contained within the self-contained unit. In some embodiments, the self-contained unit further comprises a battery unit. In some embodiments, the battery unit is configured to power the self-contained unit for at least about 10 hours without use of other power sources. In some embodiments, the self-contained unit further comprises an algae reactor, wherein the algae reactor is configured to convert at least a portion of a waste generated by the at least one water-growing organism into food for the at least one water-growing organism. In some embodiments, the algae reactor is configured to stack on one or more additional algae reactors contained within the self-contained unit. In some embodiments, the region comprises a volume from about 19 m³ to about 25 m³. In some embodiments, the region comprises a volume from about 10 m³ to about 25 m³. In some embodiments, the one or more water-growing organisms comprise shrimp. In some embodiments, the one or more water-growing organisms comprise fish. In some embodiments, the one or more water-growing organisms comprise algae. In some embodiments, the one or more water-growing organisms comprise seaweed. In some embodiments, the self-contained unit further comprises a monitor configured for detection of one or more pathogenic microorganisms. In some embodiments, the self-contained unit does not comprise a raceway. In some embodiments, the self-contained unit further comprises a regulated feeding unit. In some embodiments, the regulated feeding unit is regulated by one or more sensors housed within the self-contained unit.

In another aspect, the present disclosure provides a system, comprising: (a) a plurality of self-contained units configured for growth of at least one water-growing organism: (b) a storage bay: (c) a harvesting station: (d) a cleaning station: (e) a stocking station; and (f) an automated system configured to move the plurality of self-contained units between the storage bay, the harvesting station, the stocking station, and the cleaning station.

In some embodiments, the system further comprises a non-transitory computer readable medium comprising instructions that, when executed, direct the plurality of self-contained units between the storage bay, the harvesting station, the stocking station, and the cleaning station. In some embodiments, the storage bay, the harvesting station, the stocking station, and the cleaning station are located within a single tower. In some embodiments, the storage bay and a second storage bay are stacked vertically such that a plurality of self-contained units are stored in a vertically stacked configuration. In some embodiments, the storage bay and a second storage bay are stacked horizontally such that a plurality of self-contained units are stored in a horizontally stacked configuration. In some embodiments, the storage bay is configured to couple to an umbilical portion of a self-contained unit of the plurality of self-contained units. In some embodiments, the umbilical portion is configured to transmit one or more of electrical power, gasses, liquids, or any combination thereof. In some embodiments, the system further comprises a system configured to generate saltwater from freshwater. In some embodiments, saltwater is not discharged during the operation of the system. In some embodiments, the plurality of self-contained units is at least about 100 self-contained units. In some embodiments, the plurality of self-contained units is at least about 25 self-contained units. In some embodiments, the system comprises a plurality of storage bays, a plurality of harvesting stations, a plurality of stocking stations, or a plurality of cleaning stations.

In another aspect, the present disclosure provides a method, comprising: (a) providing a first self-contained unit and a second self-contained unit each configured for growth of at least one water-growing organism: (b) growing a first water-growing organism in the first self-contained unit, wherein the growing comprises providing a first feed source to the first water-growing organism: (c) growing a second water-growing organism in the second self-contained unit, wherein the first water-growing organism is a feed source for the second water-growing organism; and (d) regulating the providing the first feed source based on one or more of a biomass of the first water-growing organism, a biomass of the second water-growing organism, a behavioral feature of the first water-growing organism, a behavioral feature of the second water-growing organism, or any combination thereof.

In some embodiments, the regulating comprises use of one or more sensors configured to observe the first self-contained unit or the second self-contained unit, the first water-growing organism, the second water-growing organism, or any combination thereof. In some embodiments, the sensors comprise at least one of temperature sensors, pH sensors, dissolved gas sensors, turbidity sensors, nitrate sensors, nitrite sensors, nitrate sensors, ammonia sensors, phosphate sensors, cameras, sensors to measure the presence or absence of bacterial species, or any combination thereof. In some embodiments, the regulating comprises use of a machine learning algorithm. In some embodiments, the machine learning algorithm comprises a computer vision algorithm. In some embodiments, the method further comprises transporting the first water-growing organism from the first self-contained unit to the second self-contained unit. In some embodiments, the first self-contained unit and the second self-contained unit are a same self-contained unit. In some embodiments, the method further comprises providing an additional water-growing organism to the first self-contained unit or the second self-contained unit. In some embodiments, the additional water-growing organism consumes waste from one or more of the first water-growing organism or the second water-growing organism. In some embodiments, the first water-growing organism is algae or seaweed. In some embodiments, the second water-growing organism is shrimp. In some embodiments, the second water-growing organism is fish.

In another aspect, the present disclosure provides a method, comprising: (a) providing a self-contained unit configured for the growth of a water-growing organism; and (b) growing the water-growing organism with a time to maturity of the water-growing organism of less than a time to maturity of a water-growing organism grown outside of the self-contained unit.

In some embodiments, the water-growing organism is a shrimp. In some embodiments, the time to maturity is less than about 80 days. In some embodiments, the time to maturity is less than about 55 days. In some embodiments, the water-growing organism brought to maturity with a feed conversion rate of at most about 1.3. In some embodiments, the water-growing organism brought to maturity with a feed conversion rate of at most about 1. In some embodiments, the self-contained unit does not comprise a raceway.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 is a flowchart of a method, according to an embodiment of the present disclosure.

FIG. 2 is a flowchart of a method, according to an embodiment of the present disclosure.

FIG. 3 shows a computer system that is programmed or otherwise configured to implement methods provided herein.

FIGS. 4A-4B are examples of a front and side view of an algae reactor, respectively.

FIG. 5 is an example of a self-contained unit.

FIG. 6 is an example of a system comprising a plurality of self-contained units and a plurality of storage bays.

FIGS. 7A-7D are different views of an algae reactor, according to some embodiments.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

As used herein, the term “water-growing organism” generally refers to an organism which substantially grows in water. A water-growing organism may grow exclusively in water. A water-growing organism may grow partially in water. A water-growing organism may spend a portion of its life in water. A water-growing organism may be a fish (e.g., a gilled aquatic animal). A water-growing organism may be a shellfish (e.g., an invertebrate with an exoskeleton, a mollusk, a crustacean, an echinoderm, etc.). A water-growing organism may be an aquatic or semi-aquatic plant. Non-limiting examples of water-growing organism include shrimp, lobster, crab, krill, oyster, mussel, scallop, clam, cod, mackerel, salmon, trout, swordfish, shark, tuna, mullet, bass, mahi-mahi, anchovy, haddock, bream, pollack, snapper, carp, catfish, pike, trench, eel, crappie, squid, octopus, algae, seaweed, seagrass, plankton, and kelp.

In another aspect, the present disclosure provides a self-contained unit. The self-contained unit may comprise a region configured for growth of at least one water-growing organism. The growth may be at a feed conversion rate of at least about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.5, 3.0, 4.5, 5.0, or more. The growth may be at a feed conversion rate of at most about 5.0, 4.5, 4.0, 3.5, 3.0, 2.5, 2.0, 1.9, 1.8, 1.7, 1.6, 1.5, 1.4, 1.3, 1.2, 1.1, 1.0, 0.9, 0.8, 0.7, 0.6, 0.5, 0.4, 0.3, 0.2, 0.1, or less. The growth may be at a feed conversion rate in a range as defined by any two of the previous values. For example, the feed conversion rate may be at a rate of about 0.8 to 1.2. The feed conversion rate may be a ratio of the weight of a water-growing organism to the weight of feed added to the growth region containing the water-growing organism. For example, a feed conversion rate of 1 can be had where 100 kilograms of feed were added to a growth chamber and 100 kilograms of shrimp were harvested from the growth chamber. A feed conversion rate of less than 1 may mean that other food sources were used by the water-growing organism as food during its growth. For example, a presence of algae in the growth region can supplement food added to the growth region. In this example, a shrimp in the growth region can eat both feed added to the region as well as algae growing in the region. The region may comprise a volume of at least about 5, 6, 7, 8, 9, 10, 11, 12, 13, 14, 15, 16, 17, 18, 19, 20, 21, 22, 23, 24, 25, 26, 27, 28, 29, 30, 40, 50, or more cubic meters. The region may comprise a volume of at most about 50, 40, 30, 29, 28, 27, 26, 25, 24, 23, 22, 21, 20, 19, 18, 17, 16, 15, 14, 13, 12, 11, 10, 9, 8, 7, 6, 5, or fewer cubic meters. The region may comprise a volume as defined by any two of the proceeding values. For example, the region may comprise a volume from about 19 to 25 cubic meters.

The self-contained unit may not comprise a raceway. The systems and methods of the present disclosure may provide a variety of benefits over raceway-based growth units. For example, a raceway can provide strong currents within the raceway, which can slow growth of a water-growing organism due to energy expenditure from swimming. In this example, the energy the organism uses for swimming in a raceway can instead go towards growth of the organism. Another example of a benefit over raceway-based system may be that the water in a raceway is too deep. Additionally, raceway-based systems can be more difficult to clean than the systems described elsewhere herein. For example, the presence of a central partition in a raceway-based system can provide an obstacle to cleaning as well as a site for accumulation of debris. In another example, eddy currents created by the raceway can cause deposits of waste to form in the unit. Similarly, the raceway may increase a difficulty of harvesting a water-growing organism from the unit. For example, a net-based harvesting method can be complicated by the multiple regions present in a raceway. Raceway-based systems may also be more complicated and costly to construct and can add additional weight to a unit. The increased weight may result in increased difficulty preparing vertically stacked designs as described elsewhere herein. Raceway-based systems may also provide incomplete mixing of gas into the water of the unit, which can reduce efficiency of the unit.

The self-contained unit may further comprise an umbilical portion. The umbilical portion may be configured for exchange between inside the self-contained unit and outside the self-contained unit. The umbilical portion may be configured for delivery of one or more of electrical energy, gasses, solids, liquids, or any combination thereof. For example, the umbilical portion can connect to an electrical system and serve to provide energy for the operation of the self-contained unit. In another example, the umbilical portion can be configured to permit a flow of oxygen into the self-contained unit and a flow of degassed nitrogen and carbon dioxide out of the self-contained unit.

The self-contained unit may further comprise a lid. The lid may be configured to contain the region. For example, the lid can be configured to be positioned on top of the region and function to separate the region from outside the self-contained unit. The lid may be configured to keep water, other liquids, and gasses within the self-contained unit. The lid may comprise insulation. The insulation may be configured to aid in maintenance of the temperature of the region of the self-contained unit. The insulation may comprise mineral wools (e.g., fiberglass), natural fibers (e.g., cellulose), polymers (e.g., polystyrene, polyurethane, etc.), or any combination thereof. The lid may comprise a heater and/or a cooler. The heater and/or cooler may be configured to aid in a temperature maintenance of the region. The heater may comprise a resistive heater, an inductive heater, a thermoelectric heater, or the like, or any combination thereof. The cooler may comprise an evaporative cooler, a compressive cooler, a thermoelectric cooler, or the like, or any combination thereof. The heater and/or cooler may be configured to maintain a liquid (e.g., water) within the region at a temperature of at least about 0.1, 0.5, 1, 1.5, 2, 2.5, 3, 3.5, 4, 4.5, 5, 6, 7, 8, 9, 10, or more degrees Celsius from a set point temperature. The heater and/or cooler may be configured to maintain a liquid (e.g., water) within the region at a temperature of at most about 10, 9, 8, 7, 6, 5, 4.5, 4, 3.5, 3, 2.5, 2, 1.5, 1, 0.5, 0.1, or less degrees Celsius from a set point temperature. The heater and/or cooler may be configured to maintain a liquid within the region in a temperature range around a set point temperature as defined by any two of the proceeding values.

The lid may comprise one or more lights. The lights may be configured to provide light as defined by a photoperiod (e.g., an on-off cycle, a wavelength cycle, etc.). For example, the lights may be configured to provide different illumination conditions (e.g., luminescent flux, wavelengths, etc.) at different times. The photoperiod may comprise an illumination time of at least about 4, 5, 6, 7, 8, 9, 10, 11, 12, 13, 14, 15, 16, 17, 18, 19, 20, or more hours of light per day. The photoperiod may comprise an illumination time of at most about 20, 19, 18, 17, 16, 15, 14, 13, 12, 11, 10, 9, 8, 7, 6, 5, 4, or less hours of light per day. The spectral composition of the light may change throughout the photoperiod. For example, the light can transition from white light to a variety of combinations of red and blue light. The lights may generate light with a wavelength of about 350 nm to about 850 nm. The lights may generate light with a wavelength of about 350 nm to about 400 nm, about 350 nm to about 450 nm, about 350 nm to about 500 nm, about 350 nm to about 550 nm, about 350 nm to about 600 nm, about 350 nm to about 650 nm, about 350 nm to about 700 nm, about 350 nm to about 750 nm, about 350 nm to about 800 nm, about 350 nm to about 850 nm, about 400 nm to about 450 nm, about 400 nm to about 500 nm, about 400 nm to about 550 nm, about 400 nm to about 600 nm, about 400 nm to about 650 nm, about 400 nm to about 700 nm, about 400 nm to about 750 nm, about 400 nm to about 800 nm, about 400 nm to about 850 nm, about 450 nm to about 500 nm, about 450 nm to about 550 nm, about 450 nm to about 600 nm, about 450 nm to about 650 nm, about 450 nm to about 700 nm, about 450 nm to about 750 nm, about 450 nm to about 800 nm, about 450 nm to about 850 nm, about 500 nm to about 550 nm, about 500 nm to about 600 nm, about 500 nm to about 650 nm, about 500 nm to about 700 nm, about 500 nm to about 750 nm, about 500 nm to about 800 nm, about 500 nm to about 850 nm, about 550 nm to about 600 nm, about 550 nm to about 650 nm, about 550 nm to about 700 nm, about 550 nm to about 750 nm, about 550 nm to about 800 nm, about 550 nm to about 850 nm, about 600 nm to about 650 nm, about 600 nm to about 700 nm, about 600 nm to about 750 nm, about 600 nm to about 800 nm, about 600 nm to about 850 nm, about 650 nm to about 700 nm, about 650 nm to about 750 nm, about 650 nm to about 800 nm, about 650 nm to about 850 nm, about 700 nm to about 750 nm, about 700 nm to about 800 nm, about 700 nm to about 850 nm, about 750 nm to about 800 nm, about 750 nm to about 850 nm, or about 800 nm to about 850 nm. The lights may generate light with a wavelength of about 350 nm, about 400 nm, about 450 nm, about 500 nm, about 550 nm, about 600 nm, about 650 nm, about 700 nm, about 750 nm, about 800 nm, or about 850 nm. The lights may generate light with a wavelength of at least about 350 nm, about 400 nm, about 450 nm, about 500 nm, about 550 nm, about 600 nm, about 650 nm, about 700 nm, about 750 nm, or about 800 nm. The lights may generate light with a wavelength of at most about 400 nm, about 450 nm, about 500 nm, about 550 nm, about 600 nm, about 650 nm, about 700 nm, about 750 nm, about 800 nm, or about 850 nm. The lights may comprise one or more of incandescent lights, light emitting diodes (LEDs), arc lamps (e.g., mercury vapor lamps, sodium vapor lamps, halogen lamps, etc.), gas discharge lights (e.g., fluorescent lights), or the like, or any combination thereof.

The lid may comprise one or more sensors. The sensors may be as described elsewhere herein. The sensors may be configured to measure conditions within the self-contained unit. For example, the sensors may be configured to measure conditions within the growth region. The sensors may comprise one or more digital sensors. The sensors may comprise one or more analogue sensors. The sensors may be configured to measure water quality (e.g., turbidity, pH, organic molecule content, chemical composition, etc.), air quality (e.g., composition of an air layer above the growth region), video images, still images, acoustic information (e.g., air waves, sonar, hydrophonic waves, etc.), or the like, or any combination thereof. The lid may comprise one or more latches configured to attach the lid to the body of the self-contained unit. The latches may be configured in arrays of latching mechanisms (e.g., hinged latches, pin locks, etc.). The lid may be configured to generate a temporary seal with the unit. The seal may be gas-tight, liquid tight, or a combination thereof. The lid may comprise attachment points for latches contained within the body of the self-contained unit. The lid may comprise attachment points configured to permit attachment to a lift configured to remove the lid from the self-contained unit. The lid may be connected to the self-contained unit such that the lid may be opened and held in place. For example, the lid can be connected to the body by a hinge, and a series of external supports can keep the lid open during cleaning.

The self-contained unit may comprise a pump. The pump may be contained within the self-contained unit. The pump may be configured to circulate water within the self-contained unit. For example, the pump can move water around the self-contained unit to prevent stagnation. The pump can be configured to aerate the water within the self-contained unit. For example, the pump can be connected to an eductor to aerate water as it is pumped through the pump. The pump may be configured to distribute water within the self-contained unit. For example, the pump can output water throughout the growth region of the self-contained unit. The pump may be configured to exchange water from the self-contained unit with water outside of the self-contained unit. The pump may be in fluidic communication with a heater and/or cooler. The heater and/or cooler may be as described elsewhere herein. The pump may be connected to a plurality of output nozzles within the self-contained unit. The pump may be connected to at least about 5, 10, 15, 20, 25, 30, 35, 40, 45, 50, or more output nozzles. The pump may be connected to at most about 50, 45, 40, 35, 30, 25, 20, 15, 10, 5, or fewer output nozzles.

The self-contained unit may comprise a battery unit. The battery unit may comprise a plurality of battery cells. The battery unit may comprise rechargeable batteries. The battery unit may comprise one or more of lithium-ion batteries, lead-acid batteries, flow batteries, aluminum-ion batteries, metal-air batteries, molten-salt batteries, nickel-cadmium batteries, nickel-metal hydride batteries, or the like, or any combination thereof. The battery unit may be configured to maintain operation of the self-contained unit in an absence of a connection to an electrical grid. For example, during movement of the self-contained unit between storage bays in a vertically stacked tower, the battery unit can maintain the pumps and lights within the self-contained unit until the unit is reconnected to a power delivery system through an umbilical connection. The battery unit may be configured to power the self-contained unit for at least about 0.1, 0.5, 0.75, 1, 1.5, 2, 2.5, 3, 4, 5, 6, 7, 8, 9, 10, or more hours. The battery unit may be configured to power the self-contained unit for at most about 10, 9, 8, 7, 6, 5, 4, 3, 2.5, 2, 1.5, 1, 0.75, 0.5, 0.1, or less hours. The battery unit may be connected to a transformer and/or current controlling device. The transformer and/or current controlling device may be configured to convert the output of the battery to a current and frequency used by the self-contained unit. For example, the output of the battery can be transformed to power the sensors in the unit. The battery unit may be detachable from the self-contained unit. For example, the battery unit can be removed from the self-contained unit during cleaning of the self-contained unit and reattached after cleaning.

The self-contained unit may comprise a regulated feeding unit. The regulated feeding unit may be regulated by time (e.g., time-based feeding), sensor data (e.g., physical condition sensors as described elsewhere herein, camera images, biomass estimations, etc.), or the like, or any combination thereof. For example, the regulated feeding unit can determine based on the behavior of the water-growing organisms that a feeding is needed. In another example, the regulated feeding unit can begin looking for a predetermined amount of biomass in the self-contained unit after a predetermined time has passed since the last feeding. The regulated feeding unit may be configured to dispense one or more different types of feed. For example, the regulated feeding unit can be configured to dispense one feed type for a plant within the self-contained unit and another type of feed for a shrimp in the self-contained unit. The regulated feeding unit may be configured to automatically dispense food, semi-automatically dispense food, or manually dispense food. The regulated feeding unit may comprise a storage space (e.g., a hopper, a tank, etc.). The storage space may be configured to store food that can then be drawn into the self-contained unit. The food may be drawn using a conveyor, pipes, pumps, augers, pressure, or the like, or any combination thereof. The regulated feeding unit may comprise sensors configured to measure properties of the food. Examples of properties include, but are not limited to, weight of dispensed food, weight of food remaining in the storage space, water content of the food, bacterial content of the food, viscosity of the food, or the like. The food dispensed by the regulated feeding unit may be formulated as to reduce a loss of nutrients from the food into the self-contained unit. For example, the food can be formulated to reduce leeching of nutrients out of the food and into the water. By reducing the loss of nutrients from the food, the feed conversion rate of the self-contained unit can be improved. The food may be configured for easier consumption by the water-growing organisms.

The self-contained unit may comprise a nitrification moving bed bioreactor (NMBB). The NMBB may be contained within the self-contained unit. For example, the NMBB may be placed within the self-contained unit. The NMBB may be in fluidic communication with the self-contained unit. For example, the NMBB may be connected to the self-contained unit via a system of pipes. The NMBB may comprise a containment vessel configured to enclose biofiltration media and/or one or more fluidizing aeration sources. The biofiltration media may comprise one or more of polymers (e.g., plant-based polymers, artificial polymers, etc.), minerals (e.g., silica, other oxides, etc.), carbon (e.g., activated charcoal, etc.), or the like, or any combination thereof. The biofiltration media may comprise a surface area of at least about 10, 50, 100, 200, 300, 400, 500, 600, 700, 800, 900, 1,000, 1,250, 1,500, 1,750, 2,000, 2,250, 2,500, or more square meters per gram or square meters per cubic meter. The biofiltration media may comprise a surface area of at most about 2,500, 2,250, 2,000, 1,750, 1,500, 1,250, 1,000, 900, 800, 700, 600, 500, 400, 300, 200, 100, 50, 10, or less square meters per gram. The biofiltration media may comprise a surface area in a range as defined by any two of the proceeding values. The biofiltration media may comprise an oxygen concentration of at least about 0.00001, 0.00005, 0.0001, 0.0005. 0.001, 0.005, 0.01, 0.05, 0.1, 0.5, 1, 2, 3, 4, 5, 6, 7, 8, 9, 10, 15, 20, 25, or more percent or milligrams per liter. The biofiltration media may comprise an oxygen concentration of at most about 25, 20, 15, 10, 9, 8, 7, 6, 5, 4, 3, 2, 1, 0.5, 0.1, 0.05, 0.01, 0.005, 0.001, 0.0005, 0.0001, 0.00005, 0.00001, or less percent or milligrams per liter. The biofiltration media may comprise an oxygen concentration in a range as defined by any two of the proceeding values. The biofiltration media may have a neutral or substantially neutral buoyancy in water. The biofiltration media may have a negative or substantially negative buoyancy in water (e.g., the biofiltration media may sink in water). The biofiltration media may have a positive or substantially positive buoyancy in water (e.g., the biofiltration media may float in water). The biofiltration media may be configured to enhance growth of one or more microbes on the surface of the biofiltration media. For example, the biofiltration media may be configured with a surface conducive to growth of a bacteria. The biofiltration media may be configured to inhibit growth of one or more microbes on the surface of the biofiltration media. For example, the biofiltration media can be configured with antimicrobial compounds on its surface.

The containment vessel may comprise one or more of polymers (e.g., plastics, polyethylene, polytetrafluoroethylene, etc.), metals (e.g., iron, steel, aluminum, zinc, etc.), natural fibers (e.g., wood), or the like, or any combination thereof. The containment vessel may be configured to prevent movement or loss of the biofiltration media. The containment vessel may be configured to permit a fluid flow through the containment vessel. For example, the containment vessel may comprise pipes, permeable walls (e.g., walls with holes, walls with permeable membranes), or the like, or any combination thereof. The containment vessel may be configured to permit fluid flow through the biofiltration media. For example, the containment vessel can be configured to permit flow of water contaminated with waste from a growth region to the biofiltration media for removal of the waste. The containment vessel may have a weight of at least about 50, 100, 200, 300, 400, 500, 600, 700, 800, 900, 1,000, 1,250, 1,500, 1,750, 2,000, or more pounds. The containment vessel may have a weight of at most about 2,000, 1,750, 1,500, 1,250, 1,000, 900, 800, 700, 600, 500, 400, 300, 200, 100, 50, or less pounds. In some cases, the containment vessel has a volume of at most about 0.5, 1, 2, 3, 4, 5, 6, 7, 8, 9, 10, or more cubic meters (m³). The containment vessel may be opaque or substantially opaque. The growth of algae within the containment vessel may be decreased or stopped by blocking most of the light that is incident on the containment vessel. Doing so can improve the performance of the biofiltration media by preventing fouling or other buildup of algae. The containment vessel may comprise an aeration device. The aeration device may be configured to facilitate water flow within and through the containment vessel, facilitate mass transfer within or through the containment vessel, maintain predetermined conditions (e.g., aerobic conditions), or the like, or any combination thereof. The aeration device may comprise one or more hoses (e.g., hoses configured to be gas impermeable), aeration hoses (e.g., semi-permeable hoses configured to release a portion of a gas traveling through the hose), ceramic aeration emitters (e.g., porous ceramics configured to bubble out gasses), venturi emitters or eductors, other gas bubblers configured to introduce gasses into a liquid (e.g., to increase oxygen saturation within the liquid), or the like, or any combination thereof. The containment vessel may be configured to facilitate a flow of water through the vessel and across the surface area of the media.

The self-contained unit may comprise a plurality of water-growing organisms. The self-contained unit may comprise a plurality of different types of water-growing organisms. The different types of water-growing organisms may comprise water-growing plants, water-growing animals, other water-growing organisms as described elsewhere herein, or the like, or any combination thereof. For example, the self-contained unit can comprise algae, seaweed, shrimp, and filter feeding fish. The self-contained unit may comprise at least about 1, 2, 3, 4, 5, 6, 7, 8, 9, 10, or more different types of water-growing organisms. The self-contained unit may comprise at most 10, 9, 8, 7, 6, 5, 4, 3, 2, or fewer different types of water-growing organisms. The number of different types of water-growing organisms in the self-contained unit may vary over time. For example, as one type of water-growing organism grows in the self-contained unit, it can consume and extinct all of a second type of water-growing organism within the self-contained unit. A water-growing organism of the water-growing organisms may be configured to subsist on waste generated by a second water-growing organism of the water-growing organisms. For example, a filter feeding fish can subsist on waste generated by shrimp in the self-contained unit. In another example, bacteria can subsist on waste generated by shrimp and fish within the self-contained unit. In this example, the bacteria can convert nitrogenous waste into diatomic nitrogen. The self-contained unit may comprise a microbiome comprising a plurality of water-growing organisms. The microbiome may be configured to be self-contained. The microbiome may be a predetermined microbiome. For example, the microbiome can be selected by a human for optimal stability and resistance to disease. The presence of a full microbiome in the self-contained unit may provide benefits over a monoculture system such as reduced susceptibility to disease, increased growth speeds, improved feed conversion efficiencies, ant the like. The microbiome may be monitored. For example, regular testing of the microbial composition of the unit can be performed. In another example, the microbiome can be monitored for one or more pathogenic microorganisms. If the microbiome is found to be different from a predetermined microbiome, remediation may be performed. For example, additional beneficial microbes may be introduced to combat a presence of harmful microbes. In another example, antibiotics may be administered. The self-contained unit may be stocked at a high biomass stocking density. Such high stocking density may improve output per unit area of the self-contained unit. Such high stocking density may also increase the importance of proper microbiome management, as an imbalance or pathogen in a high density unit may have a greater impact that one in a low density unit. The stocking density may be at a density of 1, 5, 10, 15, 20, 25, 30, 40, 50, 60, 70, 80, 90, 100, or more kilograms of water-growing organisms per m³. The stocking density may be at a density of at most about 100, 90, 80, 70, 60, 50, 40, 30, 25, 20, 15, 10, 5, or fewer kilograms of water-growing organisms per m³.

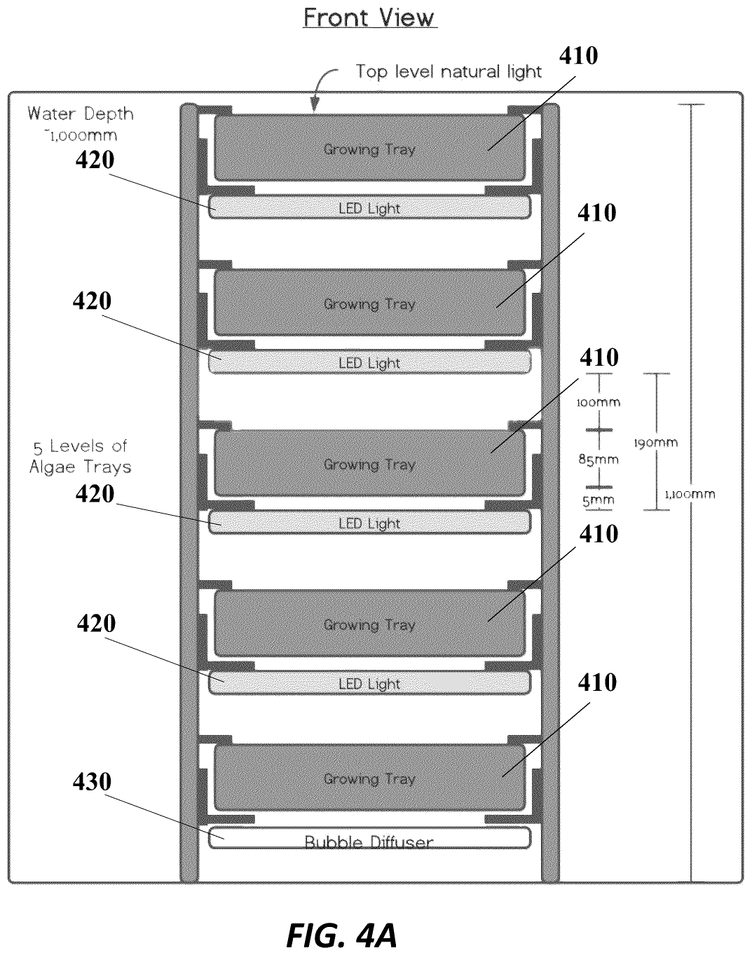

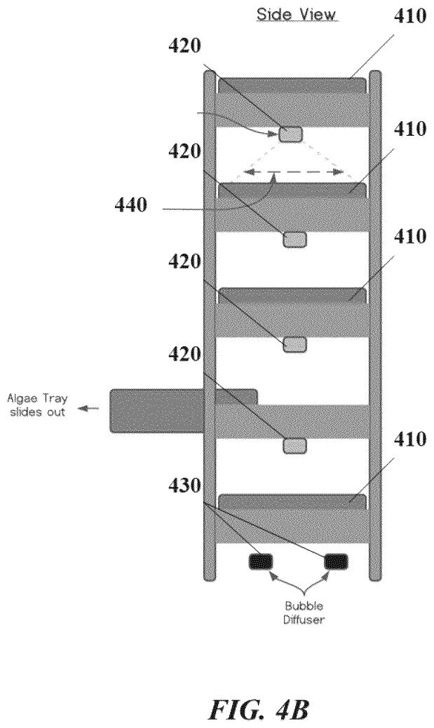

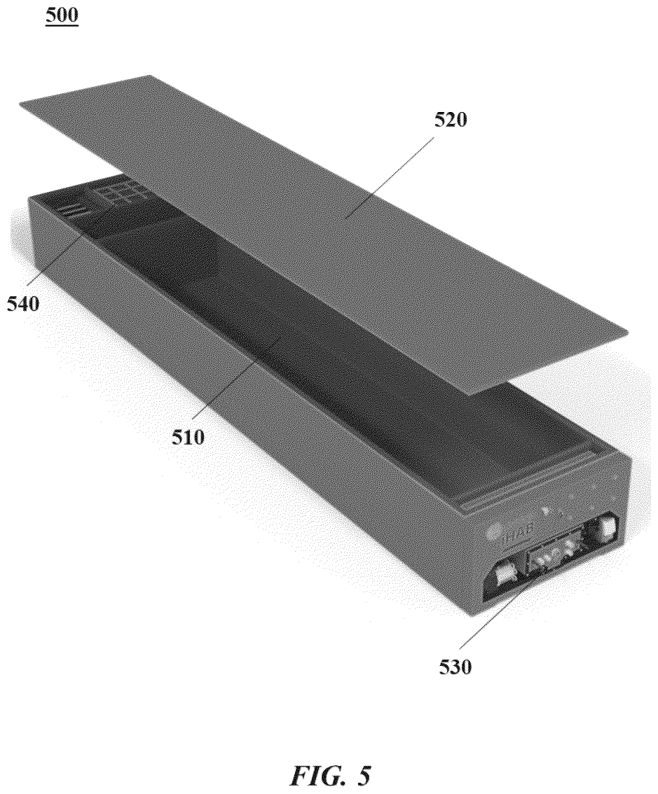

The self-contained unit may comprise an algae reactor. FIGS. 4A-4B are examples of a front and side view of an algae reactor, respectively. A non-limiting example of an algae that can be grown in an algae reactor is Caulerpa Lentillifera. The algae reactor may be configured to convert at least a portion of a waste generated by the at least one water-growing organism into food for the at least one water-growing organism. For example, the algae can utilize waste from shrimp and uneaten shrimp feed in the self-contained unit for its own growth. The algae reactor may be configured to convert at least a portion of a waste generated by the at least one water-growing organism into increased algae biomass. The increased biomass may be used as food for the at least one water-growing organism, harvested as food for another organism (e.g., food for human consumption), harvested for another non-food use (e.g., as biomass for conversion into biofuels), or the like, or any combination thereof. The algae reactor may be configured to contain a plurality of growth beds. The algae reactor may be configured to stack on one or more growth beds contained within the self-contained unit. The algae reactor may be configured to stack at least about 2, 3, 4, 5, 6, 7, 8, 9, 10, or more growth beds. The algae reactor may be configured to stack at most about 10, 9, 8, 7, 6, 5, 4, 3, or fewer growth beds. In the example of FIGS. 4A-4B, 5 growth beds are show stacked on one another. The algae reactor may comprise one or more algae growth beds 410. The growth beds may be configured to contain algae as it is growing in the reactor. The growth beds may comprise a solid bottom. The growth beds may comprise a bottom with one or more holes (e.g., a screen bottom). The growth beds may comprise polymer (e.g., plastics), metals (e.g., aluminum, zinc, stainless steel), or the like, or any combination thereof. As noted, the growth beds may be configured to be removable from the algae reactors. The growth beds may comprise materials configured to contain and/or facilitate growth of the algae. For example, the growth beds may comprise a porous bedding material configured to facilitate growth of a root system, mesh, or other retention material to restrain the growth of the algae to the growth bed, or the like. The growth beds may be configured to stack on one or more other beds contained within the self-contained unit.

The algae reactor may comprise one or more lights 420. The lights may be configured to aid in the growth of the algae. The lights may comprise one or more of incandescent lights, fluorescent lights, light emitting diodes, or the like, or any combination thereof. The lights may be oriented above growth beds for the algae to grow in. In some cases, the lights may be placed adjacent or proximate to the growth beds for the algae to grow in. The lights may be configured to provide white light, red light, blue light, green light, ultraviolet light, or any combination thereof. For example, the lights can be configured to provide white, blue, and red light. The lights may be configured to provide different light based on a predetermined schedule or predetermined wavelength combination recipe. For example, the lights can be configured to provide white light for a period, blue light for a second period, red light for a third period, and a combination of red and blue light for a fourth period. The lights may be placed behind an optional diffuser element 440. The diffuser element may be configured to diffuse the light over the algae bed to provide a more uniform illumination of the algae. The diffuser element may comprise a semi-transparent plastic diffuser or other light transmissive material. Different algae reactors may independently contain or not contain a diffuser element. The algae reactor may comprise one or more bubble diffusers 430. The bubble diffusers may be configured to bubble one or more gasses up through the algae reactors. For example, carbon dioxide can be bubbled through the algae reactors to improve a growth of the algae in the reactors. Dimensions are provided for example only.

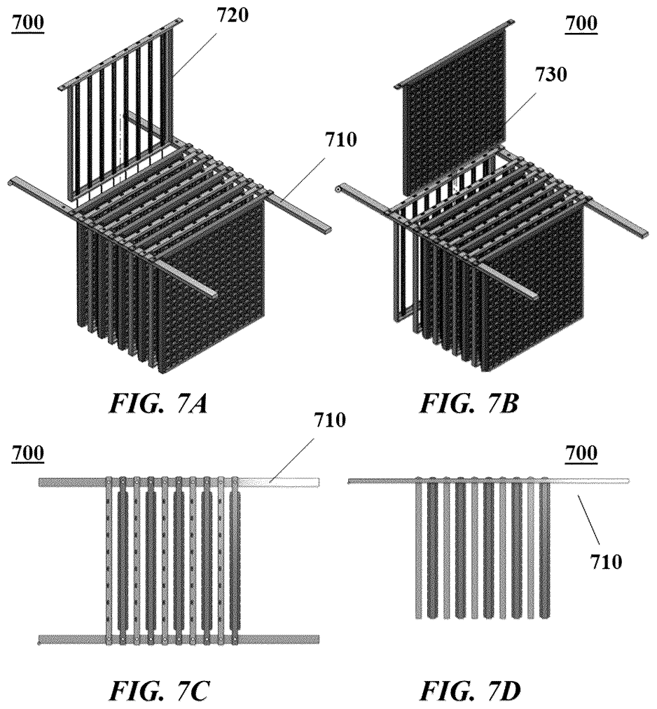

FIGS. 7A-7D are different views of an algae reactor 700, according to some embodiments. FIGS. 7A-7B show an isometric view of an algae reactor 700, while FIG. 7C shows a top view and FIG. 7D shows a side view of the same reactor 700. One or more lights (e.g., lights as described elsewhere herein) can be attached to a light frame 720, which can, in turn, be attached to a support strut 710. The algae reactor may comprise at least about 1, 2, 3, 4, 5, 6, 7, 8, 9, 10, 11, 12, 13, 14, 15, 20, or more light frames. The light frames may be interspersed with one or more growth beds 730. The one or more growth beds may be as described elsewhere herein. The algae reactor may comprise at least about 1, 2, 3, 4, 5, 6, 7, 8, 9, 10, 11, 12, 13, 14, 15, 20, or more growth beds. Each growth bed and/or light frame may be individually removable from the algae reactor. For example, if a light frame is no longer operating at capacity, the light frame can be removed and a new light frame can be installed in the algae reactor. In this way, the components of the algae reactor can be easily changed or replaced, which can improve the functioning of the reactor.

In some cases, the light frames can receive electrical power from the support struts. For example, the light frame can plug into a power delivery system contained within the support strut. In some cases, the light frames can comprise self-contained power. For example, the light frames can comprise a battery, which can be charged upon removal of the light frame from the algae reactor.

FIG. 5 is an example of a self-contained unit 500 as described elsewhere herein. The self-contained unit may comprise a growth region 510, a lid 520, an umbilical portion 530, additional devices (e.g., an NMBB, an algae reactor, a pump, etc.) 540, or any combination thereof.

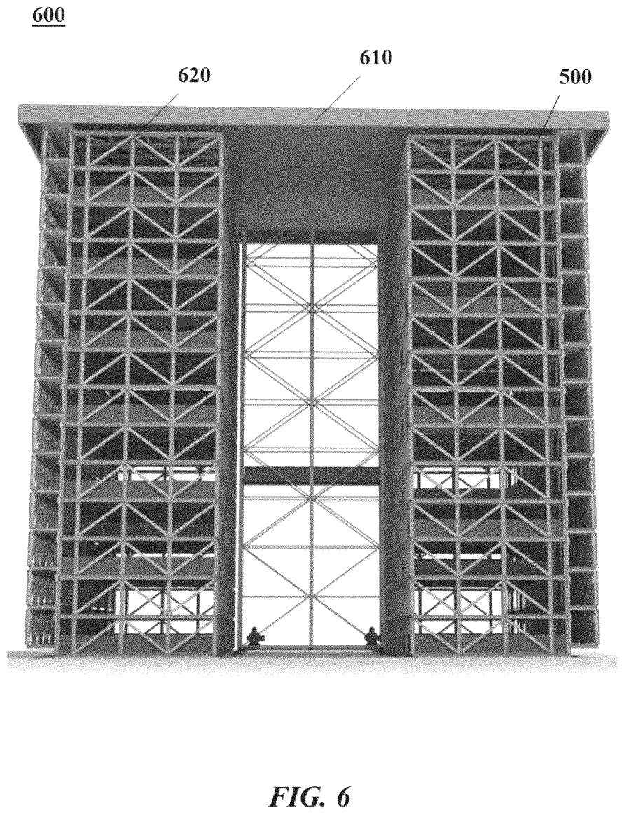

In another aspect, the present disclosure provides a system. The system may comprise a plurality of self-contained units. The plurality of self-contained units may be self-contained units as described elsewhere herein. The plurality of self-contained units may be configured for growth of at least one water-growing organism. The system may comprise a storage bay. The system may comprise a harvesting station. The system may comprise a cleaning station. The system may comprise a stocking station. The system may comprise a central hatchery or nursery system. The system may comprise an automated system configured to move the plurality of self-contained units between the storage bay, the harvesting station, the stocking station, and/or the cleaning station. The storage bay, the harvesting station, the stocking station, and the cleaning station may be located within a single tower. For example, an array of storage bays can be placed above and/or adjacent to cleaning stations, stocking stations, central hatcheries or nurseries, and harvesting stations. FIG. 6 is an example of a system 600 comprising a plurality of self-contained units 500 and a plurality of storage bays 620. The plurality of storage bays may be within a single vertical tower 610.

The system may comprise a plurality of storage bays configured for storage of one or more self-contained units. The system may comprise at least about 1, 5, 10, 25, 50, 75, 100, 150, 200, 250, or more storage bays. The system may comprise at most about 250, 200, 150, 100, 75, 50, 25, 10, 5, or fewer storage bays. The storage bays may comprise a space sized to fit the one or more self-contained units. For example, a storage bay can have enough room for a single self-contained unit to be placed in the storage bay. In another example, the storage bay can have enough room for two self-contained units to be placed side by side. The storage bays may comprise a connector configured to interact with an umbilical portion of self-contained unit. The umbilical portion may be as described elsewhere herein. For example, a storage bay can comprise a plug configured to connect to the umbilical portion of a self-contained unit and supply electrical energy, gasses, and water to the self-contained unit. The system may comprise a number of self-contained units and storage bays such that, on average, at least about 50, 60, 70, 80, 90, or more percent of the storage bays contain a self-contained unit. The system may comprise a number of self-contained units and storage bays such that, on average, at most about 90, 80, 70, 60, 50, or less percent of the storage bays contain a self-contained unit. The plurality of storage bays may be stacked vertically such that a plurality of self-contained units are stored in a vertically stacked configuration. For example, the plurality of storage bays can be placed on top of one another in a tower. The plurality of storage bays may be stacked horizontally such that a plurality of self-contained units are stored in a horizontally stacked configuration. For example, the plurality of storage bays can be placed next to one another in a tower. The plurality of storage bay's may be stacked both vertically and horizontally such that a plurality of self-contained units are stored in a grid configuration. For example, the plurality of storage bays can be placed in a tower that forms a two-dimensional grid of storage bays. In another example, the plurality of storage bays can be placed in a radial array to form a circular tower. The system may comprise at least about 10, 20, 30, 40, 50, 60, 70, 80, 90, 100, 125, 150, 175, 200, 250, 300, 400, 500, or more self-contained units, harvesting stations, and/or cleaning stations. The system may comprise at most about 500, 400, 300, 250, 200, 175, 150, 125, 100, 90, 80, 70, 60, 50, 40, 30, 20, 10, or fewer self-contained units, harvesting stations, and/or cleaning stations.

The harvesting station may be configured for harvesting one or more of the water-growing organisms. The harvesting may comprise collecting the one or more water-growing organisms from the self-contained unit. The collecting may comprise use of one or more filters, nets, pipelines, or the like, or any combination thereof. For example, a net may be drawn through the self-contained unit to collect the water-growing organism within the net. The harvesting station may be configured to separate the one or more water-growing organisms from one or more additional water-growing organisms. For example, the harvesting station can separate shrimp from aquatic plants in the self-contained unit. The harvesting station may be configured to prepare the one or more water-growing organisms for sale. The preparation may comprise euthanizing the one or more water-growing organisms, processing the one or more water-growing organisms, packing the one or more water-growing organisms, or the like, or any combination thereof. The processing may comprise removing internal and/or external organs from the one or more water-growing organisms. For example, the internal organs and external shell of shrimp can be removed in the harvesting station. The harvesting station may be configured to cohort or sort the water-growing organisms by size, weight, appearance, other criteria, or any combination thereof. For example, the harvesting station can comprise a shrimp size grading device to separate harvested shrimp into different sizes or weights.

The cleaning station may be configured to clean the self-contained unit after the one or more water-growing organisms are harvested from the unit. The cleaning station may comprise washing equipment (e.g., pressure washing equipment, mechanical scrubbers, mechanical agitators, etc.), sanitizing equipment (e.g., soaps, antibacterial solutions, steam, etc.), or the like, or any combination thereof. The cleaning station may be automated. For example, the cleaning station may clean the unit without aid of a human. The cleaning station may be semi-automated. For example, a person can clean the physical debris from the unit, and the cleaning station can automatically sanitize the unit. The cleaning station may be manual. For example, a person can both clean and sanitize the unit.

The stocking station may be configured to add water, other additives (e.g., nutrients, salts, etc.), or a combination thereof to a self-contained unit. The stocking station may be configured to add one or more water-growing organisms to the self-contained unit. For example, the stocking station can comprise counting sensors to add a predetermined number or weight of water-growing organisms to the self-contained unit. In another example, the stocking station can be configured to add immature water-growing organisms to the self-contained unit to mature. The stocking station may be configured to add one or more devices to the self-contained unit. The devices may be as described elsewhere herein. For example, the stocking station can be configured to add one or more algae reactors or MBBRs to the self-contained unit. The central hatchery and/or nursery may be configured for an initial growth of the water-growing organism. For example, shrimp can be hatched and brought to a post-larval stage in the nursery before being transferred to a self-contained unit for maturation. The water-growing organisms may be transferred from one self-contained unit to another as they mature. For example, as the water-growing organisms mature, they can be transferred to multiple self-contained units to reduce the density of the water-growing organisms.

The automated system may comprise one or more computer systems described elsewhere herein. The automated system may comprise machinery configured to move the self-contained units. For example, the automated system may comprise hydraulic lifts configured to raise self-contained units into the storage bays. The system may comprise a non-transitory computer readable medium comprising instructions that, when executed, direct the plurality of self-contained units between the storage bay, the harvesting station, and/or the cleaning station. The non-transitory computer readable medium may be implemented on a computer system as described elsewhere herein. The directing may be automatic. For example, the medium may comprise instructions that direct the movement of the self-contained units without additional input from a human. The directing may be semi-automated. For example, the medium may comprise instructions that take inputs from a human but otherwise direct the movements automatically. The directing may be manual directing. For example, a human may input the instructions for the movement of the self-contained units.

The system may comprise a system configured to generate saltwater from freshwater. Inclusion of a salination system may permit saltwater growing species to be grown away from an ocean. The salination system may be configured for introduction of salts (e.g., sodium salts, potassium salts, manganese salts, calcium salts, halide salts, carbonate salts, etc.). The salination system may be configured for addition of organic species (e.g., carbon containing chemicals). For example, a salination system can enable growth of saltwater species in inland locations. Growing saltwater species local to their consumption can reduce costs associated with transit of the species, as well as improve freshness of the final product. Combined with the stackable nature of self-contained units, this can permit growth of large amounts of high-quality products away from coastal areas, in turn reducing environmental impacts and improving overall quality. The system may be configured so as to not discharge saltwater during the operation of the system. For example, the saltwater can be contained within the self-contained units. By not discharging saltwater during operation of the system, environmental impacts caused by introduction of saltwater into non-coastal environments can be mitigated.

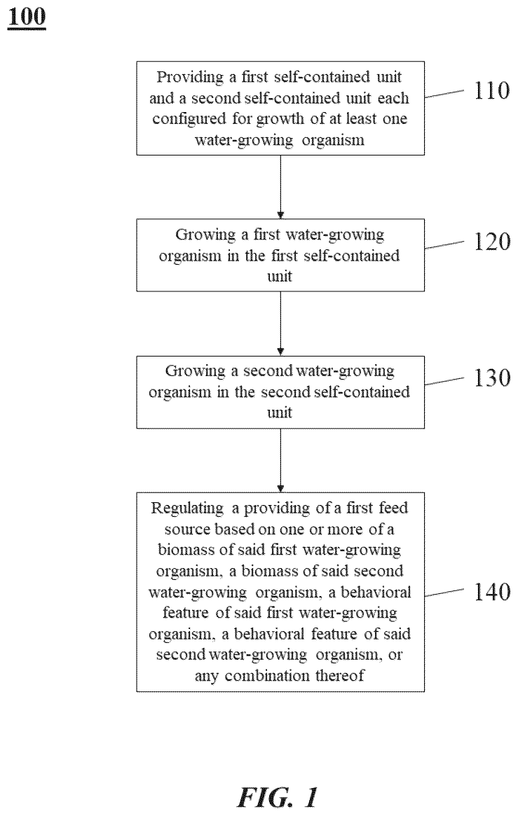

FIG. 1 is a flowchart of a method 100, according to an embodiment of the present disclosure. The method 100 may comprise providing a first self-contained unit and a second self-contained unit each configured for growth of at least one water-growing organism (110). The self-contained unit may be a self-contained unit as described elsewhere herein. In some cases, the first self-contained unit and the second self-contained unit may be the same self-contained unit. For example, a self-contained unit can be configured for growth of a plurality of water-growing organisms. The first water-growing organism may comprise algae, seaweed, or the like. The second water-growing organism may comprise shrimp, fish, or the like.

The method 100 may comprise growing a first water-growing organism in the first self-contained unit (120). The growing may comprise providing a first feed source to the first water-growing organism. The providing the first feed source may comprise use of an automated feed delivery system. For example, a feed delivery system can dispense food without intervention from a human. The food may be pellet food (e.g., solid food), liquid food (e.g., liquid nutrients), or the like, or any combination thereof.

The method 100 may comprise growing a second water-growing organism in the second self-contained unit (130). The first water-growing organism may be a feed source for the second water-growing organism. For example, the first water-growing organism can be algae and be eaten by a shrimp second water-growing organism. The second water-growing organism may subsist entirely on the first water-growing organism. For example, the second water-growing organism can eat only the first water-growing organism, and the first water-growing organism can be sufficient for the second water-growing organism. The second water-growing organism may eat other food in addition to the first water-growing organism. For example, a second water-growing organism can eat both the first water-growing organism and food pellets. The first water-growing organism may not be a feed source for the second water-growing organism. For example, the first water-growing organism can be used to reduce a waste concentration without being fed to the second water-growing organism. In this example, the first water-growing organism can be harvested and used for a non-feed purpose as described elsewhere herein.

The method 100 may comprise regulating the providing said first feed source based on one or more of a biomass of the first water-growing organism, a biomass of the second water-growing organism, a behavioral feature of the first water-growing organism, a behavioral feature of the second water-growing organism, or any combination thereof (140). The regulating may comprise use of one or more sensors. The one or more sensors may be configured to observe the first and/or self-contained units, the first and/or second water-growing organisms, or any combination thereof. For example, the sensors can be configured to observe movements of the water-growing organisms as well as the number of water-growing organisms within the self-contained units. In another example, the sensors can be configured to sense the conditions of the water in the self-contained units as well as the volume of water-growing organisms within the units. The sensors may comprise at least one of temperature sensors (e.g., thermometers, thermocouples, emission based temperature sensors, etc.), pH sensors (e.g., chemical pH sensors, electrical pH sensors, etc.), dissolved gas sensors (e.g., chemical sensors, electrical sensors, etc.), turbidity sensors (e.g., transmission sensors, laser based sensors, reflectivity sensors, etc.), nitrate sensors (e.g., chemical based sensors, electrical based sensors, etc.), nitrite sensors (e.g., chemical based sensors, electrical based sensors, etc.), nitrate sensors (e.g., chemical based sensors, electrical based sensors, etc.), phosphate sensors (e.g., chemical based sensors, electrical based sensors, etc.), cameras (e.g., photodiode arrays, complementary metal-oxide-semiconductor (CMOS) arrays, charge coupled devices (CCD), etc.), or any combination thereof.

The method 100 may comprise transporting the first water-growing organism from the first self-contained unit to the second self-contained unit. The transport may comprise use of one or more pipes. The transport may be a batch transport (e.g., the first water-growing organism is added to the second water-growing organism all at once) or a continuous transport (e.g., the first water-growing organism is added to the second water-growing organism continuously over time). The method 100 may comprise providing one or more additional water-growing organisms to the first self-contained unit or the second self-contained unit. The same one or more additional water-growing organisms may be added to both the first and the second self-contained units. For example, a same filter feeding fish can be added to the first and second self-contained units. Different additional water-growing organisms may be added to the first and the second self-contained units. For example, additional water-growing organisms can be selected for each self-contained unit based on the compatibility of the additional water-growing organisms with the first and second water-growing organisms. The additional one or more water growing organisms may consume waste from one or more of the first water-growing organism and the second water growing organism. For example, a filter feeding fish can be added to a self-contained unit with shrimp in order to reduce the amount of waste from the shrimp in the self-contained unit.

The regulating may comprise use of one or more machine learning algorithms. The machine learning algorithm may comprise a computer vision algorithm. Machine learning or computer vision algorithms implemented on computer systems described elsewhere herein or a remote server can regulate feeding of one or more water-growing organisms. For example, a machine learning algorithm can be configured to detect the number of a water growing organism in a self-contained unit. A different machine learning or computer vision algorithm can be trained to recognize behaviors of water-growing organisms and how those behaviors relate to a feeding state of the organism. Such a machine learning algorithm or computer vision algorithm can determine a biomass of a water-growing organism present in a self-contained unit.

The machine learning algorithms can be supervised, semi-supervised, or unsupervised. A supervised machine learning algorithm can be trained using labeled training inputs, e.g., training inputs with known outputs. The training inputs can be provided to an untrained or partially trained version of the machine learning algorithm to generate a predicted output. The predicted output can be compared to the known output and, if there is a difference, the parameters of the machine learning algorithm can be updated. A semi-supervised machine learning algorithm can be trained using a large number of unlabeled training inputs and a small number of labeled training inputs. An unsupervised machine learning algorithm, e.g., a clustering algorithm, can find previously unknown patterns in data sets without pre-existing labels.

One example of a machine learning algorithm that can perform some of the functions described above, e.g., estimating biomass or observing behaviors of water-growing organisms, is a neural network. Neural networks can employ multiple layers of operations to predict one or more outputs, e.g., a behavior of a water-growing organism, from one or more inputs, e.g., video feeds. Neural networks can include one or more hidden layers situated between an input layer and an output layer. The output of each layer can be used as input to another layer, e.g., the next hidden layer or the output layer. Each layer of a neural network can specify one or more transformation operations to be performed on input to the layer. Such transformation operations may be referred to as neurons. The output of a particular neuron can be a weighted sum of the inputs to the neuron, adjusted with a bias and multiplied by an activation function, e.g., a rectified linear unit (ReLU) or a sigmoid function.

Training a neural network can involve providing inputs to the untrained neural network to generate predicted outputs, comparing the predicted outputs to expected outputs, and updating the algorithm's weights and biases to account for the difference between the predicted outputs and the expected outputs. Specifically, a cost function can be used to calculate a difference between the predicted outputs and the expected outputs. By computing the derivative of the cost function with respect to the weights and biases of the network, the weights and biases can be iteratively adjusted over multiple cycles to minimize the cost function. Training can be complete when the predicted outputs satisfy a convergence condition, such as obtaining a small magnitude of calculated cost.

Convolutional neural networks (CNNs) and recurrent neural networks can be used to classify or make predictions from sensor data. CNNs are neural networks in which neurons in some layers, called convolutional layers, receive inputs from only small portions of an array of sensors. These small portions may be referred to as the neurons' receptive fields. Each neuron in such a convolutional layer can have the same weights. In this way, the convolutional layer can detect features, e.g., biomass, in any portion of the input video data.

RNNs, meanwhile, are neural networks with cyclical connections that can encode dependencies in time-series data, e.g., video frames. An RNN can include an input layer that is configured to receive a sequence of time-series inputs. An RNN can also include one or more hidden recurrent layers that maintain a state. At each time step, each hidden recurrent layer can compute an output and a next state for the layer. The next state can depend on the previous state and the current input. The state can be maintained across time steps and can capture dependencies in the input sequence. Such an RNN can be used to classify water-growing organism behaviors or masses.

One example of an RNN is a long short-term memory network (LSTM), which can be made of LSTM units. An LSTM unit can be made of a cell, an input gate, an output gate, and a forget gate. The cell can be responsible for keeping track of the dependencies between the elements in the input sequence. The input gate can control the extent to which a new value flows into the cell, the forget gate can control the extent to which a value remains in the cell, and the output gate can control the extent to which the value in the cell is used to compute the output activation of the LSTM unit. The activation function of the LSTM gate can be the logistic function.

Other examples of machine learning algorithms that can be used to classify sensor data are regression algorithms, decision trees, support vector machines, Bayesian networks, clustering algorithms, reinforcement learning algorithms, and the like.

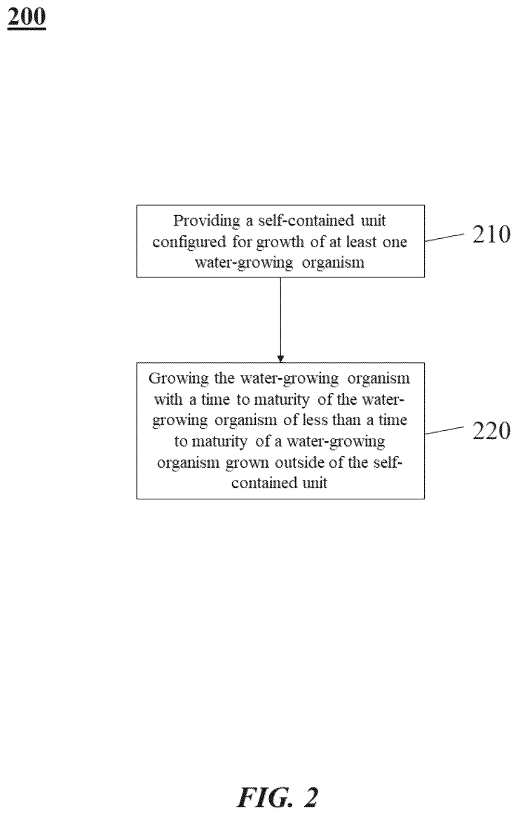

FIG. 2 is a flowchart of a method 200, according to an embodiment of the present disclosure. The method 200 may comprise providing a self-contained unit configured for the growth of a water-growing organism (210). The self-contained unit may be a self-contained unit as described elsewhere herein. For example, the self-contained unit may not comprise a raceway.

The method 200 may comprise growing the water-growing organism with a time to maturity of the water-growing organism of less than a time to maturity of a water-growing organism grown outside of the self-contained unit (220). The water-growing organism may be a shrimp. The shrimp may have a time to maturity of less than about 50, 55, 60, 65, 70, 75, 80, 85, 90, 95, 100, or more days. For example, the shrimp can grow to be a jumbo shrimp in less than about 55 days. The water-growing organism may be brought to maturity with a feed conversion rate of at least about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.5, 3.0, 4.5, 5.0, or more. The water-growing organism may be brought to maturity with a feed conversion rate of at most about 5.0, 4.5, 4.0, 3.5, 3.0, 2.5, 2.0, 1.9, 1.8, 1.7, 1.6, 1.5, 1.4, 1.3, 1.2, 1.1, 1.0, 0.9, 0.8, 0.7, 0.6, 0.5, 0.4, 0.3, 0.2, 0.1, or less. The water-growing organism may be brought to maturity with a feed conversion rate in a range as defined by any two of the previous values. For example, the feed conversion rate may be at a rate of about 0.9 to 1.1.

### Computer Systems

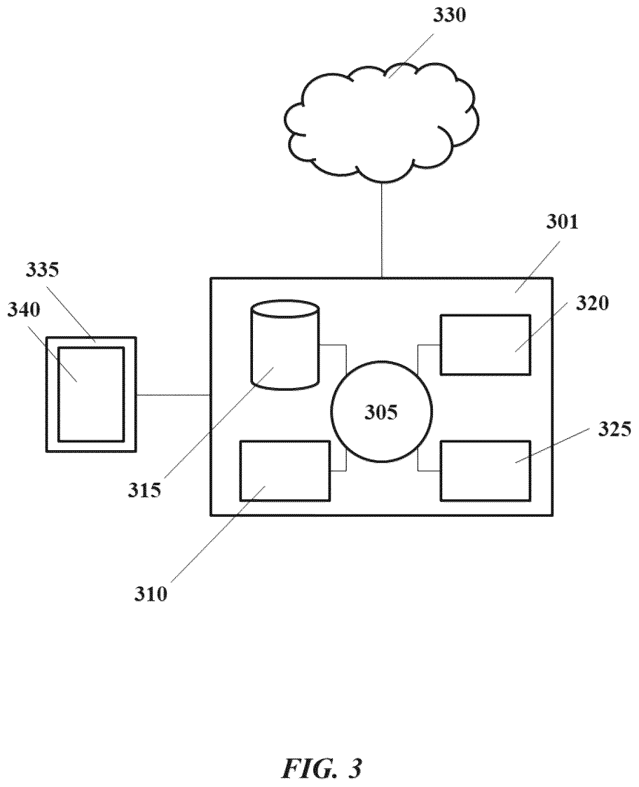

The present disclosure provides computer systems that are programmed to implement methods of the disclosure. FIG. 3 shows a computer system 301 that is programmed or otherwise configured to implement aspects of the present disclosure. The computer system 301 can regulate various aspects of the present disclosure, such as, for example, feeding of water-growing organisms, monitoring of growth regions, tracking behavior of water-growing organisms, monitoring the microbiome composition of the self-contained unit, or the like. The computer system 301 can be an electronic device of a user or a computer system that is remotely located with respect to the electronic device. The electronic device can be a mobile electronic device.

The computer system 301 includes a central processing unit (CPU, also “processor” and “computer processor” herein) 305, which can be a single core or multi core processor, or a plurality of processors for parallel processing. The computer system 301 also includes memory or memory location 310 (e.g., random-access memory, read-only memory, flash memory), electronic storage unit 315 (e.g., hard disk), communication interface 320 (e.g., network adapter) for communicating with one or more other systems, and peripheral devices 325, such as cache, other memory, data storage and/or electronic display adapters. The memory 310, storage unit 315, interface 320 and peripheral devices 325 are in communication with the CPU 305 through a communication bus (solid lines), such as a motherboard. The storage unit 315 can be a data storage unit (or data repository) for storing data. The computer system 301 can be operatively coupled to a computer network (“network”) 330 with the aid of the communication interface 320. The network 330 can be the Internet, an internet and/or extranet, or an intranet and/or extranet that is in communication with the Internet. The network 330 in some cases is a telecommunication and/or data network. The network 330 can include one or more computer servers, which can enable distributed computing, such as cloud computing. The network 330, in some cases with the aid of the computer system 301, can implement a peer-to-peer network, which may enable devices coupled to the computer system 301 to behave as a client or a server.

The CPU 305 can execute a sequence of machine-readable instructions, which can be embodied in a program or software. The instructions may be stored in a memory location, such as the memory 310. The instructions can be directed to the CPU 305, which can subsequently program or otherwise configure the CPU 305 to implement methods of the present disclosure. Examples of operations performed by the CPU 305 can include fetch, decode, execute, and writeback.

The CPU 305 can be part of a circuit, such as an integrated circuit. One or more other components of the system 301 can be included in the circuit. In some cases, the circuit is an application specific integrated circuit (ASIC).

The storage unit 315 can store files, such as drivers, libraries and saved programs. The storage unit 315 can store user data, e.g., user preferences and user programs. The computer system 301 in some cases can include one or more additional data storage units that are external to the computer system 301, such as located on a remote server that is in communication with the computer system 301 through an intranet or the Internet.

The computer system 301 can communicate with one or more remote computer systems through the network 330. For instance, the computer system 301 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 301 via the network 330.

Methods as described herein can be implemented by way of machine (e.g., computer processor) executable code stored on an electronic storage location of the computer system 301, such as, for example, on the memory 310 or electronic storage unit 315. The machine executable or machine readable code can be provided in the form of software. During use, the code can be executed by the processor 305. In some cases, the code can be retrieved from the storage unit 315 and stored on the memory 310 for ready access by the processor 305. In some situations, the electronic storage unit 315 can be precluded, and machine-executable instructions are stored on memory 310.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 301, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables: copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 301 can include or be in communication with an electronic display 335 that comprises a user interface (UI) 340 for providing, for example, a real-time health report of the status of the self-contained unit. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 305. The algorithm can, for example, be a machine learning algorithm as described elsewhere herein.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

1.-61. (canceled)

62. A system, comprising:
(a) a plurality of self-contained units configured for growth of at least one water-growing organism;
(b) a storage bay;
(c) a harvesting station;
(d) a cleaning station;
(e) a stocking station; and
(f) an automated system configured to move said plurality of self-contained units between said storage bay, said harvesting station, said stocking station, and said cleaning station.

63. The system of claim 62, further comprising a non-transitory computer readable medium comprising instructions that, when executed, direct said plurality of self-contained units between said storage bay, said harvesting station, said stocking station, and said cleaning station.

64. The system of claim 62, wherein said storage bay, said harvesting station, said stocking station, and said cleaning station are located within a single tower.

65. The system of claim 62, wherein said storage bay and a second storage bay are stacked vertically such that a plurality of self-contained units are stored in a vertically stacked configuration.

66. The system of claim 62, wherein said storage bay and a second storage bay are stacked horizontally such that a plurality of self-contained units are stored in a horizontally stacked configuration.

67. The system of claim 62, wherein said storage bay is configured to couple to an umbilical portion of a self-contained unit of said plurality of self-contained units.

68. The system of claim 67, wherein said umbilical portion is configured to transmit one or more of electrical power, gasses, liquids, or any combination thereof.

69. The system of claim 62, wherein saltwater is not discharged during the operation of the system.

70. The system of claim 62, wherein said plurality of self-contained units is at least about 25 self-contained units.

71. The system of claim 70, wherein said system comprises a plurality of storage bays, a plurality of harvesting stations, a plurality of stocking stations, or a plurality of cleaning stations.

72. The system of claim 62, wherein a self-contained unit of said plurality of self-contained units is in fluid communication with a nitrification bed bioreactor, which nitrification moving bed bioreactor comprises a containment vessel configured to enclose biofiltration media and a fluidizing aeration source.

73. The system of claim 62, wherein said at least one water-growing organism is grown at a feed conversion rate of at most about 1.3.

74. A method, comprising:
(a) providing a first self-contained unit and a second self-contained unit each configured for growth of at least one water-growing organism;
(b) growing a first water-growing organism in said first self-contained unit, wherein said growing comprises providing a first feed source to said first water-growing organism;
(c) growing a second water-growing organism in said second self-contained unit, wherein said first water-growing organism is a feed source for said second water-growing organism; and
(d) regulating said providing said first feed source based on one or more of a biomass of said first water-growing organism, a biomass of said second water-growing organism, a behavioral feature of said first water-growing organism, a behavioral feature of said second water-growing organism, or any combination thereof.

75. The method of claim 74, wherein said regulating comprises use of one or more sensors configured to observe said first self-contained unit or said second self-contained unit, said first water-growing organism, said second water-growing organism, or any combination thereof.

76. The method of claim 75, wherein said sensors comprise at least one of temperature sensors, pH sensors, dissolved gas sensors, turbidity sensors, nitrate sensors, nitrite sensors, nitrate sensors, phosphate sensors, cameras, sensors to measure the presence or absence of bacterial species, or any combination thereof.

77. The method of claim 74, wherein said regulating comprises use of a machine learning algorithm comprising a computer vision algorithm.

78. The method of claim 74, further comprising transporting said first water-growing organism from said first self-contained unit to said second self-contained unit.

79. The method of claim 74, further comprising providing an additional water-growing organism to said first self-contained unit or said second self-contained unit.

80. The method of claim 79, wherein said additional water-growing organism consumes waste from one or more of said first water-growing organism or said second water-growing organism.

81. The method of claim 74, wherein said first water-growing organism is algae or seaweed.

82. The method of claim 74, wherein said second water-growing organism is shrimp or fish.
